(19) United States
(12) Patent Application Publication
(10) Pub. No.: US 2026/0356877 A1
(43) Pub. Date: Nov. 12, 2026

(54) METHOD FOR THE SYNTHESIS OF OLIGONUCLEOTIDES WITH MODIFIED INTERNUCLEOSIDE LINKAGES

(71) Applicant: Hoffmann-La Roche Inc., Little Falls, NJ (US)

(72) Inventors: Fabio FALIVENE, Muttenz (CH); Marek Stanislaw KOMISARSKI, Bottmingen (CH); Lucca PFITZER, Gerlingen (DE); Christophe PFLEGER, Mulhouse (FR); Filippo SLADOJEVICH, Rheinfelden (CH)

(73) Assignee: Hoffmann-La Roche Inc., Little Falls, NJ (US)

(21) Appl. No.: 19/711,751

(22) Filed: Jun. 17, 2026

Related U.S. Application Data
(63) Continuation of application No. PCT/EP2024/088288, filed on Dec. 23, 2024.

(30) Foreign Application Priority Data
Dec. 27, 2023 (EP) 23220264.8

(51) Int. Cl. C07H 21/04; C07H 1/00

(52) U.S. Cl. CPC C07H 21/04; C07H 1/00

(57) ABSTRACT

The present invention relates to method for the synthesis of oligonucleotides comprising a modified internucleoside linkage.

### CROSS-REFERENCE TO RELATED APPLICATIONS

This application is a Continuation of PCT Application No. PCT/EP2024/088288, filed Dec. 23, 2024, which claims benefit of priority to EP Application No. 23220264.8 filed De-cember 27, 2023, each of which is incorporated herein by reference in its entirety.

The present invention provides a method for the synthesis of oligonucleotides with modified internucleoside linkages, in particular oligonucleotides comprising mesyl phsophoramidate and guanidine phosphoramidate linkage groups.

The mesylphosphoramidate (MsPA) linkage was recently reported by Stetsenko (PNAS, Jan. 22, 2019, vol. 116, no. 41, p. 229-1234) as an alternate to phsophorothioate (PS) for RNaseH1-mediated knock down of micro RNA targets and to modulate mRNA splicing. In the MsPA linkage, one of the non-bridging oxygen atoms in the phosphodiester linkage is replaced with methane-sulfonylamidogroup. This linkage differs from phosphoramidate linkages in that it retains negative charge on the phosphate backbone. However, it lacks the negatively charged sulfur atom that is the primary pharma-cophore for antisense oligonucleotide-protein interactions.

The synthesis of oligonucleotides with MsPA linkage uses organic azides as reagents. Azides are highly explosive compounds and there is a need for methods for the synthesis of oligonculeotides with MsPA linkages with a reduced explosion risk during the oligonucleotide synthesis.

The present invention relates to a method of preparing a modified oligonucleotide comprising contacting an oligonucleotide intermediate having a phosphite triester internucleoside linkage with an oxidizing solution comprising a oxidizing agent to form an oligonucleotide having an internucleoside linking group of formula I,

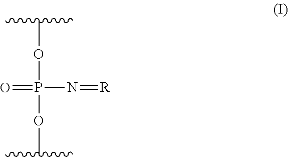

wherein R is selected from —SO₂R₃, —(NR₄R₅)₂, optionally substituted heterocycloalkyl, optionally substituted heteroaryl, wherein R₃ is selected from optionally substitute aryl, optionally substituted heterocycloalkyl, optionally substituted heteroaryl, C₁-C₆ alkoxy, C₁-C₆ alkyl, C₂-C₆ alkenyl, C₂-C₆ alkynyl, R₄ and R₅ are independently selected from C₁-C₁₂ alkyl, wherein the method is performed in a stirred bed reactor.

In an embodiment of the present invention R is selected from the group consisting of SO₂R₃,

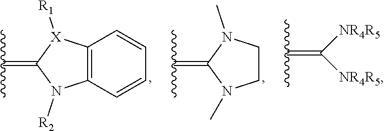

wherein X is N, O or S and R₁ and R₂ are independently selected from H or C₁-C₆ alkyl, R₃ is methyl and R₄ and R₅ are independently selected from C₁-C₁₂ alkyl.

In an embodiment of the present invention, the oligonucleotide has an internucleoside linking group selected from:

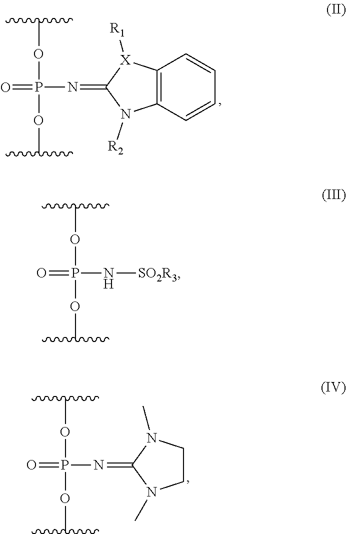

wherein R₁, R₂, and R₃ are as defined above.

In an embodiment of the present invention, the oxidizing agent is a sulfonyl oxidizing agent selected from an agent of formula N₃—SO₂R₃, preferably from mesyl azide.

In an embodiment of the present invention, the oxidizing agent is 2-azido-1,3-dimethylimidazolinum hexafluorophosphate.

In an embodiment of the present invention, the oxidizing solution comprises a stabilizing agent selected from sulfolane, triphenylphosphate and triethylphosphite.

In an embodiment of the present invention, the stirred glass bed solid phase system is quenched with triethylphosphite after completion of the oxidation step.

In an embodiment of the present invention, the stirred bed solid phase system is washed with water after the quenching with triethylphosphite.

In an embodiment of the present invention, the modified oligonucleotide comprises internucleoside linkages selected from phosphdiester, phosphorothioate, mesyl phosphoramidate and guanidine phosphoramidate linkages.

In an embodiment of the present invention, the modified oligonucleotide comprises a stereo-standard sugar moiety, a cEt sugar moiety, a 2′-MOE sugar moiety, a 2′-OMe sugar moiety, a 2′-F sugar moiety, a 2′-NMA sugar moiety, and/or a p-D-2′-deoxyribosyl sugar moiety.

In an embodiment of the present invention, the method further comprises attaching a conjugate group to form a conjugated modified oligonucleotide.

In an embodiment of the present invention, the conjugate group comprises a cell-targeting moiety.

### SHORT DESCRIPTION OF THE FIGURE

FIG. 1: Synthesis procedure for introducing mesyl phosphoramidate linkages (15mer synthesis including Staudinger oxidation)

### DEFINITIONS

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T),adenine (A) and uracil (U), cytosine (C) and guanine (G), 5-methyl cytosine (m C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. Herein, the nucleosides of the 5′-region and 3′-region each comprise a 2′-substituted furano syl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “intemucleoside linkage” or “intemucleoside linking group” means a group or bond that forms a covalent linkage between adjacent nucleosides in an oligonucleotide. As used herein “modified intemucleoside linkage” means any intemucleoside linkage other than a naturally occurring, phosphodiester intemucleoside linkage. “Phosphorothioate linkage” means a modified intemucleoside linkage in which one of the non-bridging oxygen atoms of a phosphodiester is replaced with a sulfur atom. Modified intemucleoside linkages may or may not contain a phosphorus atom. A “neutral intemucleoside linkage” is a modified intemucleoside linkage that does not have a negatively charged phosphate in a buffered aqueous solution at pH=7.0. A modified intemucleoside linkage may optionally comprise a conjugate group.

As used herein, “oligonucleotide” means a strand of linked nucleosides connected via intemucleoside linkages, wherein each nucleoside and intemucleoside linkage may be modified or unmodified. Unless otherwise indicated, oligonucleotides consist of 12-80 linked nucleosides, and optionally a conjugate group or terminal group. As used herein, “modified oligonucleotide” means an oligonucleotide, wherein at least one nucleoside (a modified nucleoside) or intemucleoside linkage (a modified intemucleoside linkage) is modified. As used herein, “unmodified oligonucleotide” means an oligonucleotide that does not comprise any nucleoside modifications or intemucleoside modifications.

Oligonucleotides are oligomeric compounds and oligonucleotides may be incorporated into oligomeric compounds having additional features. An oligonucleotide or modified oligonucleotide may comprise a linker group that links it to a solid support. The linker may be as described in Ravikumar et al., Org. Process Res. Dev. 2008, 12, 3, 399-410.

As used herein, “oligonucleotide intermediate” means a compound or portion thereof that arises during synthesis of an oligonucleotide and that will ultimately form a portion of such oligonucleotide. Oligonucleotide intermediates include, but are not limited to linked nucleosides, intemucleoside linkages, conjugate groups, and modifications described herein and precursors thereof. In certain embodiments, an oligonucleotide intermediate is a hydroxy group attached to a solid support. In certain embodiments, an oligonucleotide intermediate is a number of linked nucleosides attached to a solid support.

As used herein, “stabilizing agent” refers to a substance that when present in a solution, including but not limited to a reaction mixture, reduces the risk of explosion.

As used herein, a “standard oxidizing agent” refers to oxidizing agents well understood in the art of oligonucleotide synthesis to oxidize phosphorous intemucleoside linkages, including but not limited to basic solvents, mixtures of a basic solvent such as 3-picoline, pyridine, 2,6-lutidine with Iodine and water, mixtures of Iodine, NMI, a basic solvent, and water. Further examples and description of oxidation methods are described in WO2020236618, the disclosure of which is incorporated in its entirety herein.

As used herein, “sulfonyl oxidizing agent” means an agent that can effect transformation of a phosphite triester to a phosphoramidate. In certain embodiments, the sulfonyl oxidizing agent has a structure N₃SO₂R wherein R is as defined as for formula I. In certain embodiments. R is methyl and the sulfonyl oxidizing agent is methanesulfonyl azide (“MsNf”).

As used herein. “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S′ configuration. A cEt bicyclic sugar moiety is in the 3-D configuration.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms (“C₁₋₆-alkyl”), e.g., 1, 2, 3, 4, 5, or 6 carbon atoms. In other embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl are methyl, tert-butyl, and 2,2-dimethylpropyl.

The term “alkenyl” denotes a linear monovalent hydrocarbon chain of 2 to 7 carbon atoms or a branched monovalent hydrocarbon chain of 3 to 7 carbon atoms with at least one double bond. Exemplary alkenyl include ethenyl, 2,2-dimethylethenyl, propenyl, 2-methylpropenyl, butenyl, and pentenyl. The term “alkynyl” denotes a linear monovalent hydrocarbon chain of 2 to 6 carbon atoms or a branched monovalent hydrocarbon chain of 3 to 6 carbon atoms with at least one triple bond. Exemplary alkynyl include ethynyl, 2,2-dimethylethynyl, propynyl, 2-methylpropynyl, butynyl, and pentynyl.

The term “alkoxy” refers to an alkyl group, as previously defined, attached to the parent molecular moiety via an oxygen atom. Unless otherwise specified, the alkoxy group contains 1 to 12 carbon atoms. In some preferred embodiments, the alkoxy group contains 1 to 6 carbon atoms (“C1-6-alkoxy”). In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “aryl” refers to a monocyclic, bicyclic, or tricyclic carbocyclic ring system having a total of 6 to 14 ring members (“C₆-C₁₄-aryl”), preferably 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic, bicyclic or tricyclic, preferably monocyclic ring system having a total of 5 to 14 ring members, preferably, 5 to 12 ring members, more preferably 5 to 10 ring members, in particular 5 to 8 ring members or 5 to 6 ring members, wherein at least one ring in the system is aromatic, and at least one ring in the system contains one or more heteroatoms. Preferably, “heteroaryl” refers to a 5 to 10 membered heteroaryl comprising 1, 2, 3 or 4 heteroatoms independently selected from O, S and N. Most preferably, “heteroaryl” refers to a 5-10 membered heteroaryl, 5-8 membered heteroaryl, or 5-6 membered heteroaryl comprising 1 to 2 heteroatoms independently selected from O, S and N. Some non-limiting examples of heteroaryl include spiro[cyclopropane-1,3′-indoline](e.g., spiro[cyclopropane-1,3′-indoline]-1′-yl), 2-pyridyl, 3-pyridyl, 4-pyridyl, pyrazin-2-yl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, pyrazolo[1,5-a]pyridine, 2H-pyrazolo[4,3-b]pyridine, [1,2,4]triazolo[1,5-a]pyridine, 1H-pyrrolo[2,3-b]pyridine, imidazol-1-yl, 1H-imidazol-2-yl, 1H-imidazol-4-yl, 1H-imidazol-5-yl, oxazol-2-yl, oxazol-4-yl, oxazol-5-yl, 1,2,4-oxadiazolyl, 1,3,4-oxadiazolyl, thiazol-2-yl, thiazol-4-yl, thiazol-5-yl, pyridazin-3-yl, pyridazin-4-yl, 1,2,4-triazol-4-yl, 1,2,4-triazol-1-yl, 4H-1,2,4-triazol-3-yl, trizaol-2-yl, 2H-triazolyl, 4,5,6,7-tetrahydroindazol-2-yl, 6,7-dihydro-4H-pyrano[4,3-c]pyrazol-2-yl, thiazolyl, benzofurazan-4-yl, tetrazolyl, isoxazolyl, pyrrolyl, and morpholinyl. Particularly preferred, yet non-limiting examples of heteroaryl are pyridyl, pyrazinyl, pyrimidinyl, pyrazolyl, imidazolyl, oxazolyl, oxadiazolyl and triazolyl.

The term “heterocyclyl” refers to a saturated or partly unsaturated mono- or bicyclic, preferably monocyclic ring system of 3 to 14 ring atoms, preferably 3 to 10 ring atoms, more preferably 3 to 8 ring atoms, more preferably 3 to 6 ring atoms, in particular 3, 4, 5 or 6 ring atoms, wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidinyl, pyrrolidinyl, oxetanyl, 5-azaspiro[2.5]octan-5-yl, piperidyl, 3,3a,4,5,6,6a-hexahydro-1H-cyclopenta[c]pyrrol-2-yl, 2-azaspiro[3.3]heptan-2-yl, 2,6-diazaspiro[3.3]heptanyl, 2-azaspiro[3.4]octane, 2-azaspiro[3.5]nonan-2-yl, 1,2-dihydropyridiynl, piperidyl, and thietanyl.

### DETAILED DESCRIPTION OF THE INVENTION

The inventors of the present invention found that a stirred bed reactor can be used for the synthesis of oligonculeotides with modified backbone linkages, in particular oligonucleotides with mesylphosphoramidate internucleoside linkages.

A stirred bed reactor may comprise a mixing device and a bottom frit or filter cloth to retain the support inside the reactor when e.g. draining solvents and dissolved components.

As further examples, stirred bed reactors with a maximal inner volume of 1 to 1000 liters, of 5 to 650 liters, of 5 to 300 liters, of 10 to 600 liters, of 50 to 500 liters, or more than 1000 liters may be used. As further examples, batch reactors with a minimal inner volume of 5, 10, 30, 40, 50, 60, 70, 75, 80, 90, 100, 150, 200, 250, 300, 450, 600, 650, 700, 750, or 800 liters may be used.

The method of the present invention provides the following advantages over the prior art: Safety for Mesyl Azide (organic azides) and Staudinger reaction: these steps are performed in a glass stirred column reactor which provides controlled environment (nitrogen is released during reaction), eliminates azide-metal interaction, allows a stream nitrogen to remove hazardous acid.

The method of the present invention reduces the equivalent of mesyl azide to 10 (or even less) instead of 40 in prior art methods. The quenching of remaining mesyl azide by triethylphosphite) can be done in a solid phase synthetic step. The additional water wash of the resin removes sodium azide.

Synthesis of Internucloside Linkage Reagents and solutions are prepared in the appropriate bottles (see section 4) and attached to the respective lines of the synthesizer. NittoPhase®HL Solid Support is slurried in acetonitrile and charged to a steel column. The column is closed and connected to the synthesizer. The synthesizer is programmed according to the specified parameters (see section 3) and the run is started. After thirteen cycles (Step 1) the column is washed with acetonitrile and purged with nitrogen. The resin is dried in the oven and transferred to a stir-batch reactor.

After transferring the dried 13mer loaded resin into a stirred bed reactor, overnight pre-swelling was used to activate the resin again for the following cycles. After synthesis steps a, b and d, a wash step was performed. After completion of these two synthesis cycles, the resin was transferred again to the steel column to perform the final two couplings via automated solid phase synthesis. Resin with the bound oligonucleotide is transferred to a pressure tank and incubated with ammonia at 50 °C for 16 h. The ammonia solution is concentrated under reduced pressure, the resin is filtered off and the aqueous solution is collected. The combined crude oligonucleotide solution is analyzed for purity (RP-IP-HPLC) and content (UV260 nm) and then stored at 2-8 °C before the next step.

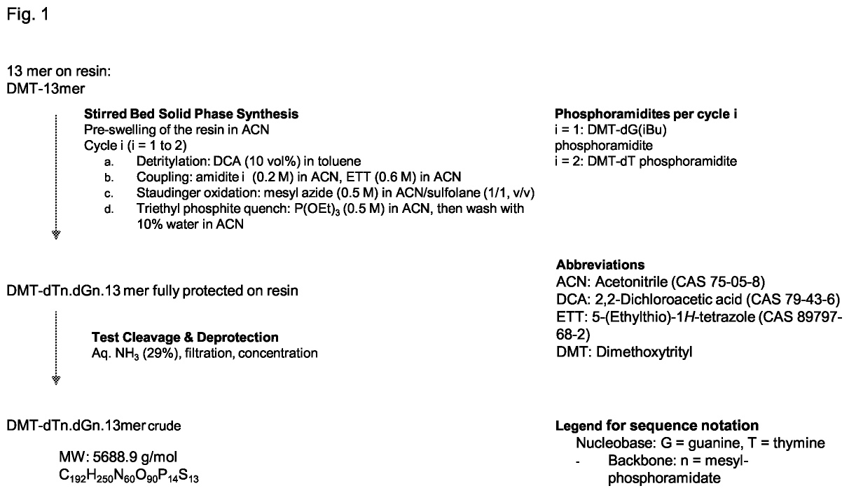

FIG. 1: Synthesis procedure for introducing mesyl phosphoramidate linkages (15mer synthesis including Staudinger oxidation)

The synthesis of oligonucleotides carrying internucleoside N-(benzoazole)-phosphoramide moieties is described in the paper of S. V. Vasilyeva et al., ACS Omega 2023, 8, 1556-1566.

The synthesis of phosphoryl guanidine oligonucleotides is described in the paper of S. A. Zhukov et al., Russian Journal of Bioorganic Chemistry, 2021, Vol. 47, No. 2, pp. 380-389.

1. A method of preparing a modified oligonucleotide comprising contacting an oligonucleotide intermediate having a phosphite triester internucleoside linkage with an oxidizing solution comprising a oxidizing agent to form an oligonucleotide having an internucleoside linking group of formula I, 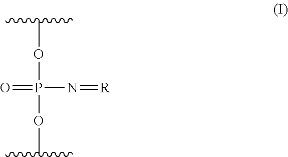
wherein R is selected from —SO₂R₃, —(NR₄R₅)₂, optionally substituted heterocycloalkyl, optionally substituted heteroaryl, wherein R₃ is selected from optionally substitute aryl, optionally substituted heterocycloalkyl, optionally substituted heteroaryl, C₁-C₆ alkoxy, C₁-C₆ alkyl, C₂-C₆ alkenyl, C₂-C₆ alkynyl, R₄ and R₅ are independently selected from C₁-C₁₂ alkyl, wherein the method is performed in a stirred bed reactor.

2. The method of claim 1, wherein R is selected from SO₂R₃, 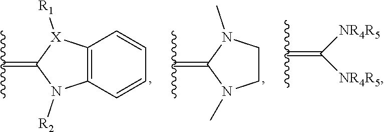
and
wherein X is N, O or S and R₁ and R₂ are independently selected from H or C₁-C₆ alkyl, R₃ is methyl and R₄ and R₅ are independently selected from C₁-C₁₂ alkyl.

3. The method of claim 1 or 2, wherein the oligonucleotide has an internucleoside linking group selected from: 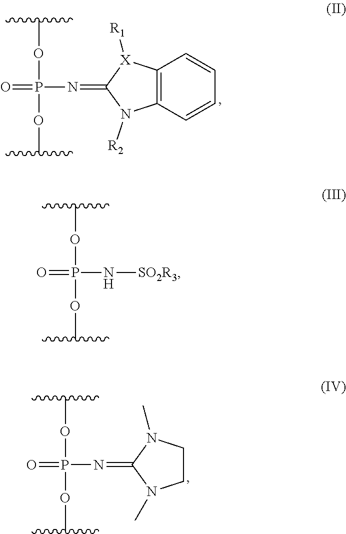
wherein R₁, R₂, and R₃ are as defined above.

4. The method of claims 1-3, wherein the oxidizing agent is a sulfonyl oxidizing agent selected from an agent of formula N₃—SO₂R₃, preferably from mesyl azide.

5. The method of claims 1-3, wherein the oxidizing agent is 2-azido-1,3-dimethylimidazolinum hexafluorophosphate.

6. The method of claims 1-5, wherein the oxidizing solution comprises a stabilizing agent selected from sulfolane, triphenylphosphate and triethylphosphite.

7. The method of claims 1-6, wherein the stirred glass bed solid phase system is quenched with triethylphosphite after completion of the oxidation step.

8. The method of claims 1-7, wherein the stirred bed solid phase system is washed with water after the quenching with triethylphosphite.

9. The method of claims 1-8, wherein the modified oligonucleotide comprises internucleoside linkages selected from phosphdiester, phosphorothioate, mesyl phosphoramidate and guanidine phosphoramidate linkages.

10. The method of claims 1-9, wherein the modified oligonucleotide comprises a stereo-standard sugar moiety, a cEt sugar moiety, a 2′-MOE sugar moiety, a 2′-OMe sugar moiety, a 2′-F sugar moiety, a 2′-NMA sugar moiety, and/or a p-D-2′-deoxyribosyl sugar moiety.

11. The method of claims 1-10, further comprising attaching a conjugate group to form a conjugated modified oligonucleotide.

12. The method of claim 11, wherein the conjugate group comprises a cell-targeting moiety.
